(19) United States
(12) Patent Application Publication
(10) Pub. No.: US 2026/0174820 A1
(43) Pub. Date: Jun. 25, 2026

(54) POLYMERS FOR INTRACELLULAR DELIVERY OF POLYNUCLEOTIDES

(71) Applicants: National University of Singapore, Singapore (SG); Shanghai Jiao Tong University, Shanghai (CN)

(72) Inventors: Xiaoyuan Chen, Singapore (SG); Pei Huang, Singapore (SG); Hongzhang Deng, Singapore (SG); Yongfeng Zhou, Shanghai (CN)

(73) Assignees: National University of Singapore, Singapore (SG); Shanghai Jiao Tong University, Shanghai (CN)

(21) Appl. No.: 19/124,838

(22) PCT Filed: Oct. 26, 2023
(86) PCT No.: PCT/SG2023/050716

§ 371 Date: Apr. 25, 2025

(30) Foreign Application Priority Data
Oct. 27, 2022 (WO) PCT/CN2022/128088

(51) Int. Cl. A61K 38/00

(52) U.S. Cl. CPC A61K 38/00; C07K 2317/76

(57) ABSTRACT

The present invention provides an ionizable polymer comprising a constitutional unit according to formula (I). wherein R1 and R2 are as defined in the specification. The ionizable polymer of the invention finds use in the intracellular delivery of polynucleotides in vitro and in vivo. The present invention also provides compositions comprising the ionizable polymer of the invention and a polynucleotide. The compositions disclosed herein find utility as medicaments, in particular as medicaments for the prevention or treatment of an infectious disease, a cancer, or a protein-deficiency disease.

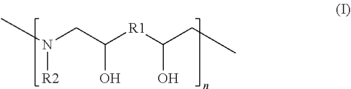

### FIELD OF INVENTION

The present invention provides an ionizable polymer comprising a constitutional unit according to formula (I). The present invention also provides compositions comprising the ionizable polymer of the invention and a polynucleotide. Compositions disclosed herein find utility as medicaments, in particular as medicaments for the prevention or treatment of an infectious disease, a cancer or a protein-deficiency disease.

### BACKGROUND OF THE INVENTION

Delivering mRNA to target cells is an important way to upregulate the expression of target proteins and has broad application prospects in mRNA vaccine, protein replacement therapy, genome editing, and cellular reprogramming. Although mRNA therapy has obvious advantages over traditional methods, it still faces many challenges during its application. One of the most critical challenges is the development of efficient and safe mRNA delivery systems.

Currently, lipid nanoparticles (LNPs) are one of the more commonly used mRNA delivery systems. LNPs are typically composed of four components, including (i) an ionizable lipid to electrostatically complex a polynucleotide, (ii) a helper phospholipid to support the structure, (iii) a cholesterol to regulate the membrane fluidity, and (iv) a polyethylene glycol (PEG)-lipid to improve colloidal stability and prolong circulation time. The requirement of the four components can be cumbersome. LNPs are also known to cause unwanted adverse effects.

Thus, there is a need for improved and/or alternative delivery vectors for polynucleotides (e.g. mRNA molecules) to enable safe and efficient intracellular delivery.

### SUMMARY OF THE INVENTION

Aspects and embodiments of the invention are described in the following numbered clauses.

1. An ionizable polymer comprising a constitutional unit according to formula (I):

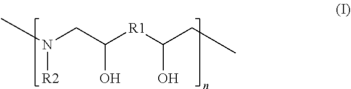

wherein
R1 represents a covalent bond or a linking moiety derived from a polyalkylene glycol; and
R2 represents a linear or branched aliphatic hydrocarbyl or a linear or branched fluorinated aliphatic hydrocarbyl group.

2. The ionizable polymer of clause 1, wherein the polyalkylene glycol is a polyethylene glycol.

3. The ionizable polymer of clause 1 or 2, wherein the polyalkylene glycol has a molecular weight of from about 200 to about 1000, optionally from about 300 to about 800, such as about 400 to about 600, e.g. about 500.

4. The ionizable polymer of any one of the preceding clauses, wherein R2 is a linear or branched alkyl or fluorinated alkyl group.

5. The ionizable polymer of any one of the preceding clauses, wherein R2 has from 1 to 25 carbon atoms.

6. The ionizable polymer of any one of the preceding clauses, wherein R2 is selected from the group consisting of butyl, hexyl, octyl, decyl, dodecyl, tetradecyl, hexadecyl and octadecyl.

7. The ionizable polymer of any one of clauses 1 to 5, wherein R2 is selected from the group consisting of difluoroethyl, pentafluoropropyl, heptafluorobutyl, nonafluoropentyl, and pentadecafluorooctyl.

8. The ionizable polymer of any one of the preceding clauses, wherein n is an integer from 1 to 10000.

9. The ionizable polymer of any one of the preceding clauses, wherein R1 is according to formula (II)

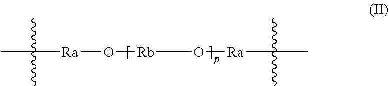

wherein,
each Ra is independently a covalent bond or a linear —C₁₋₆alkylene-;
Rb is —C₁₋₆alkylene-, said alkylene being a linear alkylene and optionally substituted by one or more groups selected from OH, NH₂, C₁₋₄alkyl, and halogen;
p is an integer from 1 to 10.

10. The ionizable polymer of any one of the preceding clauses wherein each Ra is —C₁alkylene-, and Rb is a linear and unsubstituted —C₂alkylene-.

11. The ionizable polymer of clause 1, wherein the ionizable polymer comprises a constitutional unit selected from the group consisting of:

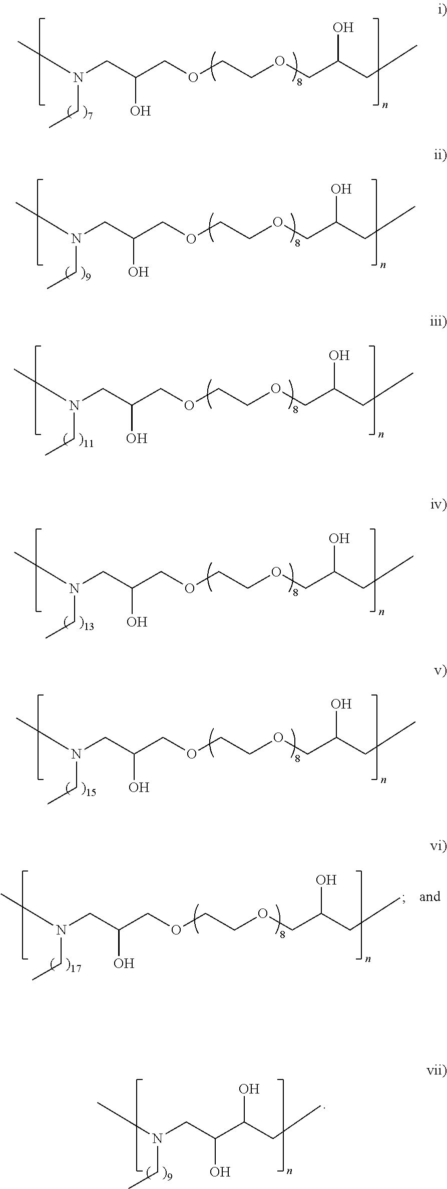

12. The ionizable polymer of clause 1, wherein the ionizable polymer comprises a constitutional unit selected from the group consisting of:

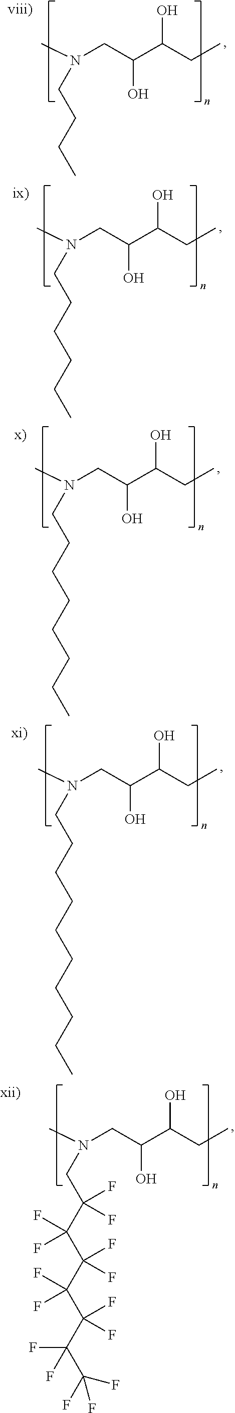

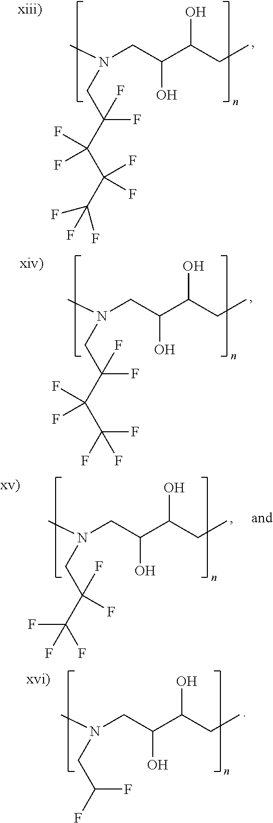

13. A composition comprising the ionizable polymer of any one of clauses 1 to 12, and a polynucleotide, wherein the polynucleotide is complexed with polymeric nanoparticles (PNP) formed from said ionizable polymer.

14. The composition of clause 13, wherein the polynucleotide is selected from the group comprising RNA and DNA, optionally wherein the polynucleotide is mRNA or siRNA.

15. The composition of clause 13 or 14, wherein:
a) R1 is derived from a polyalkylene glycol and the polynucleotide-PNP complexes are formed by mixing polynucleotide, cholesterol and ionizable polymer, optionally wherein the polyalkylene glycol is polyethylene glycol; or
b) R1 is a covalent bond and the polynucleotide-PNP complexes are formed by mixing polynucleotide and ionizable polymer.

16. The composition of any one of clauses 13 to 15, wherein the polynucleotide-PNP complex is capable of delivering said polynucleotide into a human or non-human animal cell, optionally wherein the polynucleotide is mRNA and/or wherein the human cell is a cancer cell.

17. The composition of any one of clauses 13 to 16, wherein the polynucleotide is mRNA or DNA, and where the mRNA or DNA encodes a cancer-specific antigen, an infectious disease-specific antigen, or a therapeutic protein.

18. A pharmaceutical composition comprising the composition of any one of clauses 13 to 17.

19. The pharmaceutical composition of clause 18, comprising polymeric nanoparticles (PNP) formed from a polynucleotide and an ionizable polymer, wherein:
a) R1 is a linking moiety derived from a polyethylene glycol and R2 is octadecyl, or
b) R1 is a covalent bond and R2 is decyl.

20. The pharmaceutical composition of clause 18 or 19, for use in the treatment of a cancer, wherein the mRNA or DNA encodes a cancer-specific antigen.

21. The pharmaceutical composition of clause 18 or 19, for use in the prophylaxis or treatment of an infectious disease, wherein the mRNA or DNA encodes an infectious disease-specific antigen.

22. The pharmaceutical composition of clause 21, wherein the infectious disease is a virus-related disease, and the mRNA or DNA encodes a virus-specific antigen.

23. The pharmaceutical composition of clause 18 or 19 for use in the treatment of a protein-deficiency disease, wherein the mRNA or DNA encodes a protein or peptide that is absent or non-functional in a subject to be treated.

24. A method of prophylaxis or treatment of a subject, comprising administering to the subject an efficacious amount of:
a composition of any one of clauses 13 to 17 in which the polynucleotide is an mRNA or DNA, or
a pharmaceutical composition of any one of clauses 18 to 22.

25. The method of clause 24, wherein the treatment is of a cancer in a subject and the mRNA or DNA encodes a cancer-specific antigen or a therapeutic protein.

26. The method of clause 24, wherein the prophylaxis is of a virus-related infection in a subject and the mRNA or DNA encodes a virus-specific antigen.

27. The method of clause 24, wherein the treatment or prophylaxis is of a protein-deficiency disorder in a subject, wherein the mRNA or DNA encodes a protein or peptide that is absent or non-functional in the subject.

28. Use of a composition of any one of clauses 13-17, or a pharmaceutical composition of any one of clauses 18-23 for the manufacture of a medicament for the prophylaxis or treatment of a disease selected from the group consisting of a cancer and a virus-related disease.

29. Use of a composition of any one of clauses 13-17, or a pharmaceutical composition of any one of clauses 18-23 for the manufacture of a medicament for the prophylaxis or treatment of a protein-deficiency disorder.

30. A method of producing a composition according to clause 13-17 in which the polynucleotide is mRNA or DNA, the method comprising the steps:
i) combining a monomer of formula (III) and a monomer of formula (IV) through amino-epoxy ring-opening polymerization to form an ionizable polymer comprising a constitutional unit according to formula (I); and
ii) mixing the ionizable polymer with an mRNA or DNA which encodes an antigenic polypeptide or a therapeutic protein,
   wherein,

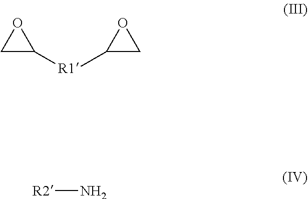

wherein,
   R1′ represents a covalent bond or a polyalkylene glycol moiety; and
   R2′ represents a linear or branched aliphatic hydrocarbyl or a linear or branched fluorinated aliphatic hydrocarbyl group.

### BRIEF DESCRIPTION OF THE FIGURES

FIG. 1 shows chemical structures of three series of polymers of the present invention (referred to herein as PHTA polymers). (a) PHTA-Cn. (b) PHTA-BCn. (c) PHTA-BFn.

FIG. 2 shows ¹H NMR spectra of PHTA-Cn polymers (CDCl₃, 400 MHz, 298 K). (a) PHTA-C8; (b) PHTA-C10; (c) PHTA-C12; (d) PHTA-C14; (e) PHTA-C16; and (f) PHTA-C18.

FIG. 3 shows size distribution of PHTA-Cn/mOVA nanovaccines before and after mOVA loading.

FIG. 4 shows zeta potential of PHTA-Cn/mOVA nanovaccines before and after mOVA loading.

FIG. 5 shows representative TEM image of PHTA-based polymeric nanoparticle (PNP).

FIG. 6 shows agarose gel electrophoresis images of PHTA-Cn/mOVA on day 1 and day 8 post mOVA loading.

FIG. 7 shows green fluorescent protein (GFP) expression in DC 2.4 cells receiving transfection of indicated formulations PHTA-Cn/mGFP (1 μg/mL mGFP) examined by confocal microscopy images.

FIG. 8 shows (a) representative flow cytometry plots showing the expression levels of GFP in DC 2.4 cells receiving transfection of the indicated formulations PHTA-Cn/mGFP (1 μg/mL mGFP); and (b) the corresponding quantitative percentages of GFP-highly-expressing cells (GFP^{high} cells) (n=3).

FIG. 9 shows (a) In vivo bioluminescence images after footpad subcutaneous injection of LNP/mFluc, PHTA-C8/mFluc and PHTA-C18/mFluc into mice, respectively. (b) Quantitative analysis of bioluminescence signals (n=5/group).

FIG. 10 shows the therapeutic schedule illustrating that B16-OVA tumor-bearing mice receiving vaccination on days 4, 7, and 10 post-tumor inoculation.

FIG. 11 shows average tumor growth curves of mice receiving indicated treatments (n=5/group).

FIG. 12 shows tumor inhibition efficiency calculated according to the tumor volumes at the endpoint compared with the PBS group (n=5/group).

FIG. 13 shows body weight curves of mice receiving indicated treatments. (n=5/group).

FIG. 14 shows ¹H NMR spectra of PHTA-BCn polymers (CDCl₃, 400 MHz, 298 K). (a) PHTA-BC4; (b) PHTA-BC6; (c) PHTA-BC8; (d) PHTA-BC10.

FIG. 15 shows agarose gel electrophoresis image of PHTA-BCn/mGFP complexes loaded with green fluorescent protein encoded mRNA (mGFP).

FIG. 16 shows the construction and characterizations of PHTA-BC10/mOVA nanovaccine. (a) scheme illustrating the construction of PHTA-BC10/mOVA nanovaccine by microfluidic mixing of PHTA-BC10 polymer and model antigen ovalbumin encoded mRNA (mOVA), (b) size of PHTA-BC10/mOVA nanovaccine before and after mOVA loading, (c) zeta potential of PHTA-BC10/mOVA nanovaccine before and after mOVA loading.

FIG. 17 shows semi-quantitative analysis of mean fluorescence intensity (MFI) of green fluorescent protein (GFP) expression in DC 2.4 receiving transfection of indicated formulations PHTA-BCn/mGFP (1 μg/mL mGFP).

FIG. 18 shows the tumor inhibition effect of PHTA-BC10/mOVA as a therapeutic cancer vaccine. (a) Average tumor growth curves of mice receiving indicated treatments (n=5/group). (b) Tumor inhibition efficiency calculated according to the tumor volumes at the endpoint compared with the PBS group (n=5/group).

FIG. 19 shows MALDI-TOF-MS of PHTA-BFn polymers. (a) PHTA-BF2; (b) PHTA-BF5; (c) PHTA-BF7; (d) PHTA-BF9. (e) PHTA-BF15.

FIG. 20 shows the characterizations of PHTA-BF7/mRNA complex. (a) agarose gel electrophoresis image of PHTA-BF7/mRNA complex. (b) size distribution of PHTA-BF7/mRNA complex before and after mRNA loading.

FIG. 21 shows semi-quantitative analysis of mean fluorescence intensity (MFI) of green fluorescent protein (GFP) expression in DC 2.4, RAW 264.7, HEK 293T, and PC3 cells receiving transfection of PHTA-BF7/mGFP (1 μg/mL mGFP).

FIG. 22 shows in vivo bioluminescence images of mice at 8 h and 24 h post subcutaneous injection of PHTA-BF7/mFluc into mice.

### DETAILED DESCRIPTION OF THE INVENTION

The present inventors have found that ionizable polymers comprising a constitutional unit according to formula (I) are surprisingly effective at delivering polynucleotides into cells, in vitro and in vivo, facilitating endosomal escape of the polynucleotide, and allowing for translation of proteins or peptides encoded by the polynucleotide. Using a mouse model for cancer, the present inventors have also shown that the ionizable polymers are capable of in vivo delivery of mRNA encoding a model cancer antigen (mOVA), resulting in a strong tumor inhibitory effect in the mice. The ionizable polymers were found to be surprisingly well tolerated with no apparent toxicity in the mice. Accordingly, the ionizable polymers of the present invention show great potential as delivery vectors in polynucleotide-based therapeutics, such as mRNA-based therapeutics.

Thus, the present invention provides an ionizable polymer for intracellular delivery of polynucleotides in vitro and in vivo, said ionizable polymer comprising a constitutional unit of formula (I):

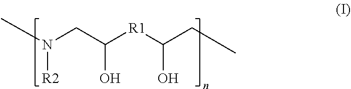

In formula (I), R1 represents a covalent bond or a linking moiety derived from a polyalkylene glycol.

The term “polyalkylene glycol” as used herein refers to polymers having a general formula HO—[R—O]ₙ—H, wherein R is an alkylene group. The hydroxyl groups are terminal groups. Common examples of polyalkylene glycols are, for example, polyethylene glycol (wherein R=a linear C₂alkylene group). The alkylene group may be unsubstituted or substituted, for example substituted by one or more groups selected from OH, NH₂, C₁₋₄alkyl, and halogen. The term “derived from a polyalkylene glycol” as used herein refers to a group within the constitutional unit of formula (I) which may be obtained from a polyalkylene glycol or a polyalkylene glycol containing compound. The term encompasses groups that comprise a polyalkylene glycol moiety.

In formula (I), n may be an integer from 1 to 10000, for example, 1 to 5000, 1 to 4000, 1 to 3000, 1 to 2000 and 1 to 1000. As will be appreciated, the ionisable polymer of the present invention can be formed in various ways, including the polymerization of monomers and/or chemical modification of one or more repeating units of a polymer precursor. Typically, it is formed by polymerization of monomers. The number of constitutional units within the ionisable polymer is therefore dependent on the degree of polymerisation, which may not be consistent between each polymer molecules within a sample. As such, in addition to the value of the integer n in formula (I), the size of the ionizable polymer of the present invention may be defined by the number average molecular weight (Mn) of the ionizable polymer in a given sample.

Typically, the ionizable polymer of the present invention has a number average molecular weight (Mn) of from about 1000 to about 10000, for example, from about 2000 to about 8000, or from about 3000 to about 7000.

In certain embodiments, the ionizable polymer of the present invention has a number average molecular weight (Mn) of about 4100, about 4700, about 4900, about 5100, about 5600 or about 6200.

In certain embodiments, R1 may comprise a polyalkylene glycol moiety that has a molecular weight of from about 200 to about 1000. For example, a molecular weight of from about 300 to about 800, such as about 400 to about 600 (e.g. about 500). For example, the polyalkylene glycol moiety may be a polyethylene glycol moiety with a molecular weight of from about 200 to about 1000. For example, a molecular weight of from about 300 to about 800, such as about 400 to about 600 (e.g. about 500).

In certain embodiments, R1 is according to formula (II):

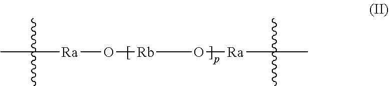

In formula (II), each Ra is independently a covalent bond or a linear —C₁₋₆alkylene-. For example, each Ra may be a covalent bond or a linear —C₁₋₆alkylene-, such as a C₁alkylene, a linear C₂alkylene, a linear C₃alkylene, a linear C₄alkylene, a linear C₅alkylene, or a linear C₆alkylene. In certain embodiments, each Ra is a C₁alkylene group. For the avoidance of doubt, when R1 is according to formula (II), R1 may still be considered to be derived from a polyalkylene glycol. In this regard, the portion of formula (II) that corresponds to the polyalkylene glycol moiety being represented by —O—[Rb—O]ₚ— in formula (II).

In formula (II), Rb is —C₁₋₆alkylene-. The alkylene is a linear alkylene and may be substituted by one or more groups selected from OH, NH₂, C₁₋₄alkyl, and halogen (e.g. fluorine, chlorine, bromine or iodine).

In formula (II), p is an integer from 1 to 1000, for example, 1 to 500, 1 to 400, 1 to 300, 1 to 200, 1 to 100, 1 to 50, 1 to 40, 1 to 30, 1 to 20, and 1 to 10. In certain embodiments, p is 1 to 10 (e.g. 1, 2, 3, 4, 5, 6, 7, 8, 9 or 10). In certain exemplary embodiments, p is 8.

In formula (I), R2 represents a linear or branched aliphatic hydrocarbyl or a linear or branched fluorinated aliphatic hydrocarbyl group.

The term “hydrocarbyl” as used herein refers to univalent substituents containing only hydrogen and carbon atoms. The term encompasses substituents that are branched or unbranched, saturated or unsaturated, cyclic, polycyclic or noncyclic species. Specific examples of hydrocarbyls include alkyl, cycloalkyl, alkenyl, alkadienyl, cycloalkenyl, cycloalkadienyl, aryl, and alkynyl groups. The hydrocarbyl group may be an aliphatic hydrocarbyl. The term “aliphatic hydrocarbyl” refers to linear or branched hydrocarbon chains saturated that are fully saturated or contain one or more unsaturated units. Examples of aliphatic hydrocarbyl groups include substituted or unsubstituted alkyl, alkenyl, alkynyl groups. In certain embodiments, R2 may be a fluorinated aliphatic hydrocarbyl. The term “fluorinated aliphatic hydrocarbyl” refers an aliphatic hydrocarbyl as defined herein wherein the aliphatic hydrocarbyl is substituted by one or more fluorine atoms.

In certain embodiments, R2 may be a linear or branched C₁₋₂₅alkyl (e.g. C₁₋₁₈alkyl) optionally substituted by one or more fluorine atoms. For example, R2 may be a C₄₋₁₈alkyl (e.g. C₄₋₁₀alkyl and C₈₋₁₈alkyl). Or, for example, R2 may be a C₂₋₈alkyl, wherein said alkyl is substituted by one or more fluorine atoms (e.g. 1 to 15 fluorine atoms, for example, 2, 5, 7, 9 or 15 fluorine atoms). In certain embodiments, R2 may be an aliphatic hydrocarbyl selected from the group consisting of butyl, hexyl, octyl, decyl, dodecyl, tetradecyl, hexadecyl and octadecyl (e.g. octyl, decyl, dodecyl, tetradecyl, hexadecyl and octadecyl). In certain other embodiments, R2 may be an aliphatic hydrocarbyl selected from the group consisting of ethyl, propyl, butyl, pentyl, and octyl, wherein the aliphatic hydrocarbyl is substituted by one or more fluorine atoms (e.g. R2 may be difluoroethyl, pentafluoropropyl, heptafluorobutyl, nonafluoropentyl, or pentadecafluorooctyl).

In certain embodiments, when R1 is a linking moiety derived from a polyalkylene glycol, R2 may be an aliphatic hydrocarbyl selected from the group consisting of octyl, decyl, dodecyl, tetradecyl, hexadecyl and octadecyl. In certain other embodiments, when R1 is a covalent bond, R2 may be an aliphatic hydrocarbyl selected from the group consisting of butyl, hexyl, octyl, decyl. In certain other embodiments, when R1 is a covalent bond, R2 may be an aliphatic hydrocarbyl selected from the group consisting of ethyl, propyl, butyl, pentyl, and octyl, wherein the aliphatic hydrocarbyl is substituted by one or more fluorine atoms (e.g. R2 may be difluoroethyl, pentafluoropropyl, heptafluorobutyl, nonafluoropentyl, or pentadecafluorooctyl). Typically, the aliphatic hydrocarbyl at R2 is a linear aliphatic hydrocarbyl (e.g. a linear C₁₋₂₅alkyl).

As used herein, the term “alkyl” refers to both linear and branched chain saturated hydrocarbon groups. Examples of alkyl groups include methyl, ethyl, n-propyl, iso-propyl, n-butyl, t-butyl, i-butyl, sec-butyl, pentyl and hexyl groups. Exemplary linear alkyl groups include methyl, ethyl, n-propyl, iso-propyl, n-butyl groups. Exemplary branched alkyl groups include t-butyl, i-butyl, 1-ethylpropyl and 1-ethylbutyl groups.

As used herein, the term “aryl” refers to monocyclic or bicyclic aromatic carbocyclic groups. Examples of aryl groups include phenyl and naphthyl. In a bicyclic aromatic group, one of the rings may, for example, be partially saturated. Examples of such groups include indanyl and tetrahydronaphthyl.

As used herein, the term “halogen” refers to fluorine, chlorine, bromine or iodine. Fluorine is particularly preferred.

In certain exemplary embodiments, the ionizable polymer of the present invention comprises a constitutional unit having a structure selected from the group consisting of:

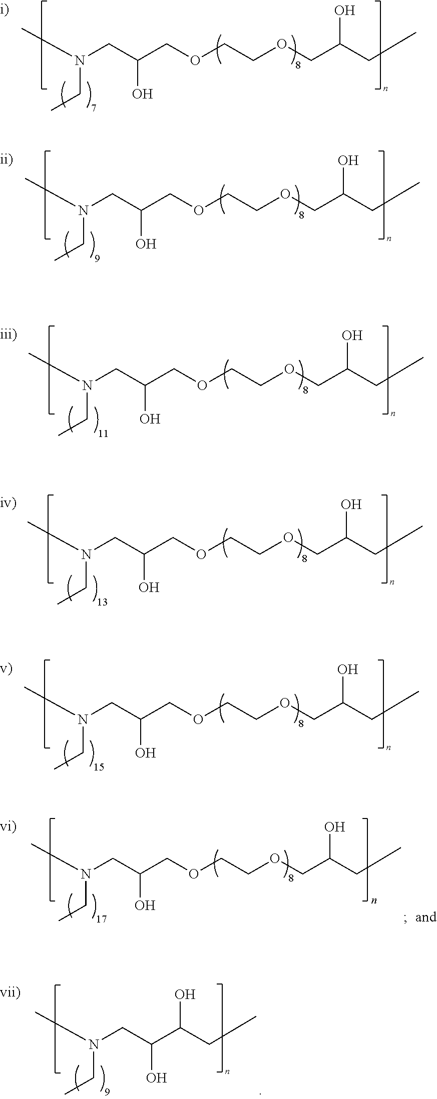

In certain other exemplary embodiments, the ionizable polymer of the present invention comprises a constitutional unit having a structure selected from the group consisting of:

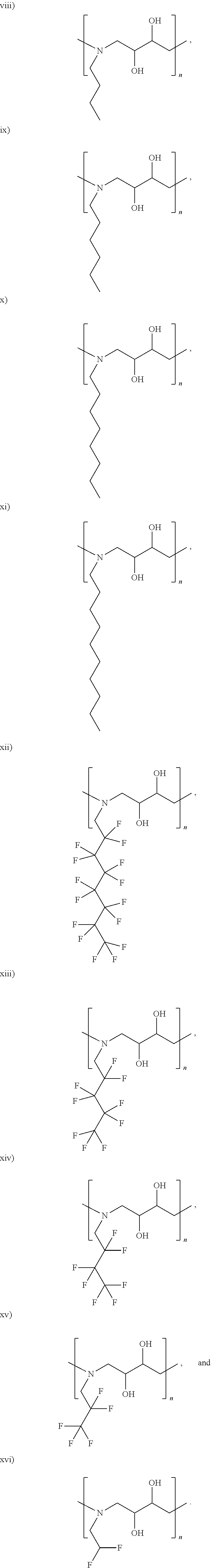

The present invention also provides an ionizable polymer produced by amino-epoxy ring-opening polymerization between a monomer according to formula (III) and a monomer according to formula (IV):

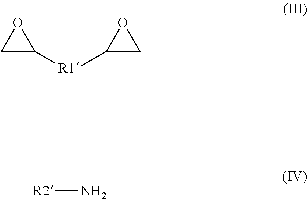

wherein, R1′ represents a covalent bond or a polyalkylene glycol moiety, and R2′ represents a linear or branched aliphatic hydrocarbyl or a linear or branched fluorinated aliphatic hydrocarbyl group.

R1′ may have a structure according to formula (IIIa):

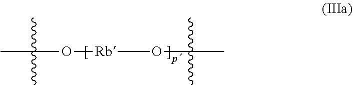

In formula (IIIa), Rb′ is —C₁₋₆alkylene-. The alkylene is a linear alkylene and may be substituted by one or more groups selected from OH, NH₂, C₁₋₄alkyl, and halogen (e.g. fluorine, chlorine, bromine or iodine). In formula (IIIa), p′ is an integer from 1 to 1000, for example, 1 to 500, 1 to 400, 1 to 300, 1 to 200, 1 to 100, 1 to 50, 1 to 40, 1 to 30, 1 to 20, and 1 to 10. In certain embodiments, p′ is 1 to 10 (e.g. 1, 2, 3, 4, 5, 6, 7, 8, 9 or 10). In certain exemplary embodiments, p′ is 8.

R2′ may be a linear or branched C₁₋₂₅alkyl (e.g. C₁₋₁₈alkyl) optionally substituted by one or more fluorine atoms. For example, R2′ may be a C₄₋₁₈alkyl (e.g. C₄₋₁₀alkyl and C₈₋₁₈alkyl). Or, for example, R2′ may be a C₂₋₈alkyl, wherein said alkyl is substituted by one or more fluorine atoms (e.g. 1 to 15 fluorine atoms, for example, 2, 5, 7, 9 or 15 fluorine atoms). In certain embodiments, R2′ may be an aliphatic hydrocarbyl selected from the group consisting of butyl, hexyl, octyl, decyl, dodecyl, tetradecyl, hexadecyl and octadecyl (e.g. octyl, decyl, dodecyl, tetradecyl, hexadecyl and octadecyl). In certain other embodiments, R2′ may be an aliphatic hydrocarbyl selected from the group consisting of ethyl, propyl, butyl, pentyl, and octyl, wherein the aliphatic hydrocarbyl is substituted by one or more fluorine atoms (e.g. R2′ may be difluoroethyl, pentafluoropropyl, heptafluorobutyl, nonafluoropentyl, or pentadecafluorooctyl).

For example, when is a R1′ is a polyalkylene glycol moiety, R2′ may be an aliphatic hydrocarbyl selected from the group consisting of octyl, decyl, dodecyl, tetradecyl, hexadecyl and octadecyl. Or, for example, when R1′ is a covalent bond, R2′ may be an aliphatic hydrocarbyl selected from the group consisting of butyl, hexyl, octyl, decyl. Or, for example, when R1′ is a covalent bond, R2′ may be an aliphatic hydrocarbyl selected from the group consisting of ethyl, propyl, butyl, pentyl, and octyl, wherein the aliphatic hydrocarbyl is substituted by one or more fluorine atoms (e.g. R2′ may be difluoroethyl, pentafluoropropyl, heptafluorobutyl, nonafluoropentyl, or pentadecafluorooctyl). Typically, the aliphatic hydrocarbyl at R2′ is a linear aliphatic hydrocarbyl (e.g. a linear C₁₋₂₅alkyl).

In certain preferred embodiments, the ionizable polymer of the present invention is produced by amino-epoxy ring-opening polymerization between a poly (ethylene glycol) diglycidyl ether or 1,3-butadiene diepoxide, and an amine selected from the group consisting of butylamine, hexylamine, octylamine, decylamine, dodecylamine, tetradecylamine, hexadecylamine, octadecylamine, and a combination of two or more thereof. In certain other preferred embodiments, the ionizable polymer of the present invention is produced by amino-epoxy ring-opening polymerization between a poly (ethylene glycol) diglycidyl ether or 1,3-butadiene diepoxide, and an amine selected from the group consisting of difluoroethylamine, pentafluoropropylamine, heptafluorobutylamine, nonafluoropentylamine, pentadecafluorooctylamine, and a combination of two or more thereof.

For example, the ionizable polymer of the present invention may be produced by amino-epoxy ring-opening polymerization between a poly (ethylene glycol) diglycidyl ether and an amine selected from the group consisting of octylamine, decylamine, dodecylamine, tetradecylamine, hexadecylamine, octadecylamine, and a combination of two or more thereof. Or, for example, the ionizable polymer of the present invention may be produced by amino-epoxy ring-opening polymerization between a 1,3-butadiene diepoxide and an amine selected from the group consisting of butylamine, hexylamine, octylamine, decylamine, and a combination of two or more thereof. Or, for example, the ionizable polymer of the present invention may be produced by amino-epoxy ring-opening polymerization between a 1,3-butadiene diepoxide and an amine selected from the group consisting of difluoroethylamine, pentafluoropropylamine, heptafluorobutylamine, nonafluoropentylamine, pentadecafluorooctylamine, and a combination of two or more thereof.

Typically, the poly (ethylene glycol) portion of the poly (ethylene glycol) diglycidyl ether has a number average molecular weight (Mn) of from about 100 to about 1000, for example, from about 200 to about 800. In certain exemplary embodiments, the poly (ethylene glycol) portion of the poly (ethylene glycol) diglycidyl ether has a Mn of about 500.

As used herein, the term “number average molecular weight” and “Mn”, when used with respect to a polymer disclosed herein, refers to the average molecule weight of a polymer that is calculated by dividing the total weight of polymer sample by the total number of molecules in the sample. The use of the term “molecular weight” herein, with respect to a polymer, is to be understood as referring to the number average molecular weight of the given polymer. The number average molecular weight of a polymer can be determined by techniques known in the art, such as gel permeation chromatography, viscometry, mass spectrometry or particle number dependent methods such as vapor pressure osmometry, endgroup measurements or proton NMR.

The ionizable polymers of the present invention are capable of forming polymeric nanoparticles (PNPs) comprising an ionizable polymer and a polynucleotide. The polymeric nanoparticles may be formed by mixing together an ionizable polymer and a polynucleotide, and optionally cholesterol. A particular method of mixing that is suitable for forming the polymeric nanoparticles (PNPs) is microfluidic mixing. Additional or alternative methods include, but are not limited to, pipette mixing and vortex mixing. Specific methods are disclosed in the Examples section herein.

In certain embodiments, the ionizable polymer of the present invention consists essentially of constitutional units according to formula (I), as described hereinabove.

### Compositions

The present invention also provides a composition comprising an ionizable polymer of the present invention and a polynucleotide, wherein the polynucleotide is complexed with said ionizable polymer to form polymeric nanoparticles (PNP). The polymeric nanoparticles (PNP) formed from the ionizable polymer and polynucleotide typically have an average diameter of about 300 nm or less (e.g., about 200 or less, for example, an average diameter from about 10 nm to about 200 nm, from about 40 nm to about 150 nm, or from about 100 nm to about 200 nm). Polymeric nanoparticles comprising an ionizable polymer of the present invention and a polynucleotide may be referred to herein as “polynucleotide-PNP complexes” or “polymeric nanovaccines”.

The polynucleotide-PNP complexes disclosed herein are capable of delivering the polynucleotide present in the complex into a eukaryotic cell, for example a human or non-human eukaryotic cell. Typically, the polynucleotide-PNP complexes disclosed herein are used to deliver the polynucleotide into a human cell, such as a human cancer cell.

For the avoidance of doubt, the term “polynucleotide” as used herein, refers to any molecule that comprises a polymer of nucleotides. The polynucleotide may be an isolated polynucleotide molecule or a polynucleotide construct (e.g. a messenger RNA (mRNA) or plasmid DNA (pDNA)). The polynucleotide may be linear (e.g., an mRNA, or an siRNA) or circular (e.g., a plasmid). The polynucleotide may also be a double-stranded or single-stranded polynucleotide. Typically, the polynucleotide is a RNA molecule or a deoxyribonucleic acid (DNA) molecule. However, it may also be a derivative of a RNA and/or DNA, for example, it may be a peptide nucleic acid oligomer (PNA)). In certain exemplary embodiments, the polynucleotide is an mRNA molecule.

The polynucleotide encodes a protein or peptide of interest. For example, the polynucleotide may encode a protein or peptide that is a cancer-specific antigen or a protein or peptide that is an infectious disease-specific antigen, or the polynucleotide may encode a therapeutic protein or peptide (e.g. a therapeutic antibody or antibody fragment).

When the polynucleotide encodes a cancer-specific antigen, the cancer-specific antigen may be a tumor-associated antigen (TAA) or tumor-specific antigen (TSA). TAAs are immunogenic proteins or peptides that are aberrantly expressed by cancer cells, for example, TAAs can be immunogenic proteins or peptides that are expressed by both normal and cancer cells, but expressed at higher levels by cancer cells. TSA are immunogenic proteins or peptides that are expressed by cancer cells, but not normal cells. A particular example of a tumor specific antigen is a neoantigen. Particular examples of tumor associated antigens include NY-ESO-1, tyrosinase, MAGE-A3, and TPTE, MAGE-C1, MAGE-C2, TPBG 5T4, survivin, and MUC-1.

When the polynucleotide encodes an infectious disease-specific antigen, the antigen may be any immunogenic protein or peptide derived from a microbe capable of causing disease by invasion of a subject. For example, the infectious disease-specific antigen may be an immunogenic protein or peptide derived from a pathogenic bacterium, fungus, parasite, or virus. Particular examples of infectious disease-specific antigens include the spike protein of SARS-CoV-2, a haemagglutinin protein of an influenza virus, a membrane or envelope protein of zika virus, fusion protein of respiratory syncytial virus (RSV), and surface glycoproteins of a human immunodeficiency virus (HIV), ebola virus or rabies virus.

When the polynucleotide encodes a therapeutic protein or peptide, the protein or peptide may be any protein or peptide that is beneficial in the treatment or prophylaxis of any inherited or acquired disease or which improves the condition of a subject. A particular example of a therapeutic protein is a therapeutic antibody or antibody fragment. Further examples of therapeutic proteins or peptides include CRISPR associated protein (CAS), a cytokine (e.g. OX40L, IL-2, IL-12, IL-12sc, IL-15sushi, IL-23, and IL-36γ, IFNα, and GM-CSF), methylmalonyl-coenzyme A mutase, propionyl-coenzyme A (CoA) carboxylase, cystic fibrosis transmembrane conductance regulator, ornithine transcarbamylase (OTC), glycogen debranching enzyme; PTEN and p53.

In certain embodiments, the composition of the present invention further comprises cholesterol. Cholesterol present in the compositions of the present invention can facilitate intracellular delivery of the polynucleotide and PNPs into cells by regulating membrane fluidity.

The present invention also provides a method of producing a composition of the present invention, the method comprising the steps:
(i) combining a monomer of formula (III) and a monomer of formula (IV) through amino-epoxy ring-opening polymerization to form an ionizable polymer comprising a constitutional unit according to formula (I); and
(ii) mixing the ionizable polymer with an mRNA or DNA which encodes an antigenic polypeptide or a therapeutic protein.

For the avoidance of doubt, formulas (III) and (IV) in step i) are according to formulas (III) and formula (IV) as described hereinabove with respect to the ionizable polymer of the present invention. In certain embodiments, in step i), the monomer according to formula (III) is a poly (ethylene glycol) diglycidyl ether or 1,3-butadiene diepoxide, and the monomer according to formula (IV) is an amine selected from the group consisting of butylamine, hexylamine, octylamine, decylamine, dodecylamine, tetradecylamine, hexadecylamine, octadecylamine, and a combination of two or more thereof. In certain other embodiments, in step i), the monomer according to formula (III) is a poly (ethylene glycol) diglycidyl ether or 1,3-butadiene diepoxide, and the monomer according to formula (IV) is an amine selected from the group consisting of difluoroethylamine, pentafluoropropylamine, heptafluorobutylamine, nonafluoropentylamine, pentadecafluorooctylamine, and a combination of two or more thereof.

The composition comprising an ionizable polymer of the present invention and a polynucleotide may be provided as a pharmaceutical composition, optionally comprising one or more pharmaceutically acceptable excipients. Pharmaceutical compositions of the present invention may be provided as any composition suitable for parenteral (including subcutaneous, intradermal, intralymphatic, intraosseous infusion, intramuscular, intravascular (bolus or infusion), and intramedullary), intratumoral, intracerebral intraperitoneal, nasal or mouth inhalation administration. Typically, the pharmaceutical composition is one suitable for intradermal, intranasal or intravenous administration. The most suitable route of administration, and therefore most suitable form of composition, may depend, for example, on the condition of the recipient.

Pharmaceutical compositions of the invention may include aqueous or non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Pharmaceutical compositions suitable for inhalation administration include solutions in saline, which can contain, for example, benzyl alcohol or other suitable preservatives, absorption promoters to enhance bioavailability, and/or other solubilizing or dispersing agents such as those known in the art. The pharmaceutical composition may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous solutions and suspensions may be prepared from sterile powders, granules and tablets. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

In embodiments wherein the polynucleotide in the pharmaceutical composition encodes an antigenic protein or peptide (e.g. a protein or peptide that is a cancer antigen or an infectious disease-specific antigen), the pharmaceutical composition may further comprise an adjuvant. As used herein, the term “adjuvant” should be understood as any substance that enhances the immune response in a subject to an antigenic protein or peptide. Examples of adjuvants include, but are not limited to, polyinosinic acid:polycytidylic acid, incomplete Freund's adjuvant (IFA), cytokines (such as interleukin), CD40, keyhole limpet hemocyanin, Toll-like receptor Liposomes, CpG oligodeoxynucleotides, saponins, colloidal alum and lipopolysaccharide lipid A analogues.

It should be understood that in addition to the ingredients particularly mentioned above, the compositions for use in this invention may include other agents conventional in the art having regard to the type of composition in question. Otherwise, the preparation of suitable formulations may be achieved routinely by the skilled person using routine techniques and/or in accordance with standard and/or accepted pharmaceutical practice.

### Treatments

The compositions of the present invention find use as medicaments. In particular, the compositions of the present invention find use in the treatment or prophylaxis of a disease.

The type of disease that may be treated or prevented by the compositions of the present invention depends on the type of protein or peptide encoded by the polynucleotide present in the composition. For example, when the polynucleotide present in the composition encodes a cancer-specific antigen, the composition may be used in the prevention or treatment of a cancer in a subject. In this context, the composition of the invention may be considered to be a therapeutic vaccine. Examples of cancers that may be prevented or treated using a composition of the present invention include, but not limited to, melanoma, colorectal cancer, head-and-neck squamous carcinoma, and non-small cell lung cancer (NSCLC).

When the polynucleotide present in the composition encodes an infectious disease-specific antigen, the composition may be used in the prevention or treatment of an infectious disease in a subject. In this context, the composition of the invention may be considered to be a preventative or therapeutic vaccine. When the infectious disease-specific antigen is a virus-specific antigen, the composition may find use in the prevention or treatment of a virus-related disease, such as illness caused by severe acute respiratory syndrome coronavirus 2 (SARS-CoV-2), an influenza viruse, zika virus, respiratory syncytial virus (RSV), human immunodeficiency virus (HIV), ebola virus, rabies virus, cytomegalovirus (CMV). The composition of the present invention may also find use in the treatment of diseases caused by bacterial, fungal, or parasitic infections (e.g. plasmodium infection).

When the polynucleotide present in the composition encodes a therapeutic protein or peptide, the composition may be used in the treatment of an inherited or acquired disease that may be treated by the therapeutic protein or peptide. For example, when the polynucleotide encodes a therapeutic antibody that displays anticancer activity, the composition may find use in the treatment of a cancer in a subject.

The compositions of the present invention may also find use in the prevention or treatment of a protein-deficiency disease. The term “protein-deficiency disease” as used herein refers to diseases that are associated with a particular protein, or group of proteins, being absent or non-functional in a patient. In this context, the composition of the present invention may be considered a protein replacement therapy. Examples of protein-deficiency disease include methylmalonyl-coenzyme A mutase deficiency, propionyl-CoA carboxylase deficiency, cystic fibrosis, ornithine transcarbamylase deficiency, glycogen storage disease type Ill (GSD Ill), PTEN hamartoma tumor syndrome and cancers associated with p53. Thus, in certain embodiments, when the polynucleotide present in the composition encodes a protein or peptide that is absent or non-functional in a subject, the composition may find use in the treatment of a protein-deficiency disease. In certain other embodiments, the composition of the invention may comprise a polynucleotide encoding a CRISPR associated protein (CAS) and/or a guide RNA (gRNA), in such embodiments, the composition may find use in genome editing in a subject, for example genome editing to treat or prevent a protein-deficiency disease in the subject.

The amount of the composition of the present invention administered to a subject will depend on various factors, such as the severity of the condition to be treated, the particular patient to be treated, the immunogenicity of the antigenic polypeptide encoded by the polynucleotide in the composition, or the potency of the therapeutic polypeptide encoded the polynucleotide in the composition. In any event, the medical practitioner, or other skilled person, will be able to determine routinely the actual dosage, which will be most suitable for an individual patient. However, typically, the dosage administered is one that may be considered to be a “therapeutic amount” or “efficacious amount”.

The terms “therapeutic amount” or “efficacious amount” as used herein may refer to the administration of an amount of the composition of the present invention that is capable of preventing a disease or improving the condition of a subject suffering from a disease. For example, when the polynucleotide in the composition encodes an antigenic protein or peptide, a “therapeutic amount” or “efficacious amount” may be considered to be an amount of the composition capable of eliciting an immune response in a subject against the antigenic polypeptide. For example, a primary and/or a secondary immune response that prevents a subject from acquiring an infectious disease or a cancer and/or improves the condition of a subject suffering from an infectious disease or a cancer. When the polynucleotide in the composition encodes a therapeutic protein or peptide (e.g. an antibody or antibody fragment), a “therapeutic amount” or “efficacious amount” may refer to an amount of the composition that is sufficient to induce the expression of the therapeutic protein or peptide in the subject at a level that results in an improvement in the condition of the subject suffering from a disease.

In certain embodiments, the composition may be administered to a subject by intradermal, intranasal or intravenous administration.

Also disclosed herein is a kit comprising the comprising a composition of the present invention. That is to say that the kit contains a composition comprising the ionizable polymer as disclosed herein and a polynucleotide as disclosed herein, wherein the polynucleotide is complexed with polymeric nanoparticles (PNP) formed from the ionizable polymer.

Also disclosed herein is a kit comprising a compound according to formula (III) and a compound according to formula (IV) as disclosed herein, together with a polynucleotide that encodes a cancer-specific antigen, an infectious disease-specific antigen, or a therapeutic protein or peptide, as disclosed herein. Such a kit of the present invention finds used in the preparation of a composition of the present invention.

For the avoidance of doubt, a kit according to the present invention is in a form and quantity suitable for use according to the present invention. The skilled person can readily determine a quantity of each component (i.e. the ionizable polymer, compounds of formulas (III) and (IIV), and/or the polynucleotide) suitable for including in a kit of the invention and for use according to the invention.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, and the like.

Further aspects and embodiments of the invention will now be discussed by reference to the following non-limiting examples.

### EXAMPLES

### Materials and Methods

Ionizable alternating copolymers with hydroxyl tertiary amine (HTA) repeating units (referred to herein as PHTA, i.e. “polymers with hydroxyl tertiary amine”) were synthesized by amino-epoxy polymerization of diepoxide monomer 1 and amine monomer 2.

The diepoxide monomer 1 can be poly(ethylene glycol) diglycidyl ether, 1,3-butadiene diepoxide (BDE), or other diepoxide monomers. The amine monomer 2 can be alkylamines such as butylamine, hexylamine, octylamine, decylamine, dodecylamine, tetradecylamine, hexadecylamine or octadecylamine. The amine monomer 2 also can be fluorine-substituted alkylamines such as difluoroethylamine, pentafluoropropylamine, heptafluorobutylamine, nonafluoropentylamine, or pentadecafluorooctylamine. By changing the types of diepoxide monomer 1 and amine monomer 2, three representative series of PHTA polymers (i.e., PHTA-Cn, PHTA-BCn, and PHTA-BFn) were synthesized and investigated for mRNA delivery efficiency. The synthetic methods of these three series of PHTA polymers are described as follows respectively:

### Synthesis of PHTA-Cn Series

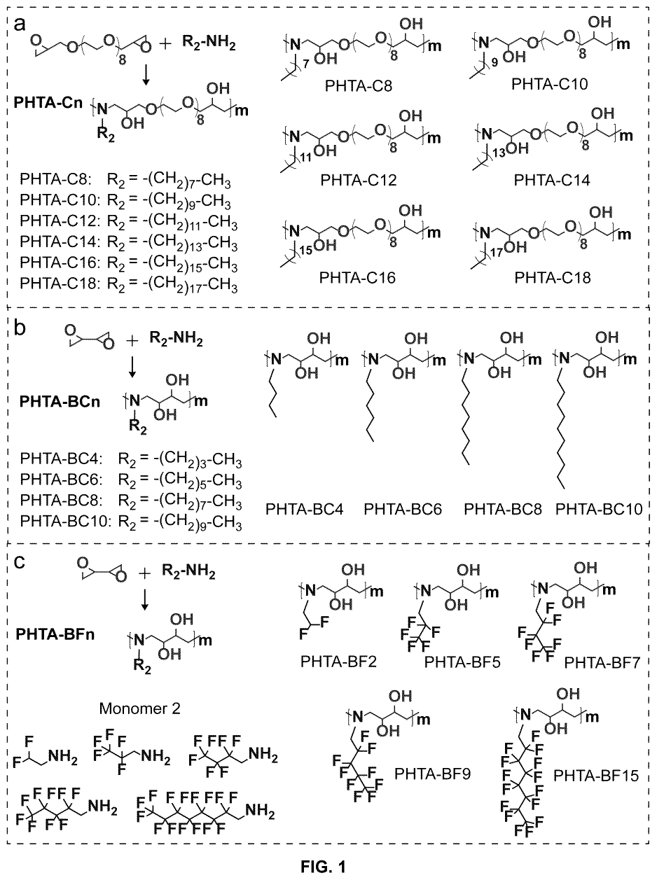

Ionizable alternating copolymers PHTA-Cn were synthesized by amino-epoxy polymerization of poly(ethylene glycol) diglycidyl ether monomer 1 and different alkylamine monomers 2 (octylamine, decylamine, dodecylamine, tetradecylamine, hexadecylamine and octadecylamine), where “n” represents the number of carbon atoms on the polymer side chain (FIG. 1a). In detail, poly(ethylene glycol) diglycidyl ether and octylamine, or decylamine, or dodecylamine, or tetradecylamine, or hexadecylamine, or octadecylamine were dissolved in propylene glycol methyl ether. The mixture was heated to reflux at 120° C. under the nitrogen atmosphere, and the reaction was stirred for 24 hours. The crude product was dialyzed against ethanol for three days to remove unreacted monomers for purification, and the final product was obtained by vacuum drying.

### Synthesis of PHTA-BCn Series

Ionizable alternating copolymers PHTA-BCn were synthesized by amino-epoxy polymerization of 1,3-butadiene diepoxide (BDE) monomer 1 and different alkylamine monomers 2 (butylamine, hexylamine, octylamine, decylamine), where “n” represents the number of carbon atoms on the polymer side chain (FIG. 1b). In detail, 1,3-butadiene diepoxide (BDE) and butylamine, or hexylamine, or octylamine, or decylamine were dissolved in ethanol and prepolymerized at room temperature for 3 hours. Then the reaction was heated to 80° C. and stirred for 24 hours at the argon atmosphere. The crude product was dialyzed against ethanol for three days to remove the unreacted monomer for purification, and the final product was obtained by vacuum drying.

### Synthesis of PHTA-BFn Series

Ionizable alternating copolymers PHTA-BFn were synthesized by amino-epoxy polymerization of 1,3-butadiene diepoxide (BDE) monomer 1 and different fluorine-substituted alkylamine monomers 2 (difluoroethylamine, pentafluoropropylamine, heptafluorobutylamine, nonafluoropentylamine, pentadecafluorooctylamine), where “n” represents the number of fluorine atoms on side chain of the polymer (FIG. 1c). In detail, 1,3-butadiene diepoxide (BDE) and difluoroethylamine, or pentafluoropropylamine, or heptafluorobutylamine, or nonafluoropentylamine, or pentadecafluorooctylamine were dissolved in ethanol and pre-polymerized at room temperature for 3 hours. Then the reaction was heated to 80° C. and stirred for 24 hours under an argon atmosphere. The crude product was dialyzed against ethanol for three days to remove the unreacted monomer for purification, and the final product was obtained by vacuum drying.

mRNA Loading and Characterization

mRNA was loaded into PHTA-based polymeric nanocarriers by microfluidic mixing technology. Specifically, mRNA was loaded into PHTA-Cn-based polymeric nanocarrier by microfluidic mixing of PHTA-Cn polymer, cholesterol, as well as mRNA, and loaded into PHTA-BCn-/PHTA-BFn-based polymeric nanocarrier by microfluidic mixing of PHTA-BCn/PHTA-BFn polymer and mRNA (without cholesterol).

In more detail, the PHTA-based polymeric nanocarriers (without mRNA) were prepared by using microfluidics mixing method. The PHTA ionizable polymers were dissolved in ethanol (if the polymers were PHTA-Cn, cholesterol was also included). The polymer-ethanol solutions were rapidly mixed with a citrate buffer via microfluidics chips, and then the mixtures were dialyzed against PBS to provide ionizable polymeric nanoparticles (in the form of nanoparticles without mRNA). The ionizable polymer/mRNA compositions were prepared by using microfluidics mixing method. The PHTA ionizable polymers were dissolved in ethanol (if the polymers were PHTA-Cn, cholesterol was also included). mRNA was diluted in citrate buffer. The polymer-ethanol solutions were rapidly mixed with mRNA citrate buffer solution via microfluidics chips, and then the mixtures were dialyzed against PBS to provide an ionizable polymer/mRNA composition (in the form of nanoparticles)

The physicochemical properties of the polymeric nanocarriers (without mRNA) and ionizable polymer/mRNA composition were characterized by dynamic light scattering (DLS), zeta potential and transmission electron microscopy (TEM). The mRNA loading capacity was evaluated by agarose gel electrophoresis.

mRNA Delivery Efficiency Evaluation

To evaluate in vitro mRNA delivery efficiency, mRNA encoding green fluorescent protein (mGFP) was loaded into PHTA-based polymeric nanocarriers and their in vitro transfection efficiency in cells were characterized by confocal microscopy images or flow cytometry. To evaluate in vivo mRNA delivery efficiency, mRNA encoding firefly luciferase (mFluc) was loaded into PHTA-based polymeric nanocarriers and their in vivo transfection efficiency in mice were characterized by an IVIS optical imaging system.

Proof of Concept Study on mRNA Therapy Delivered by PHTA-Based Polymeric Nanocarrier

To evaluate the efficacy of mRNA therapy delivered by PHTA-based polymeric nanocarrier, mRNA encoding model antigen ovalbumin (mOVA) was loaded into PHTA-based polymeric nanocarrier to prepare a polymeric mRNA vaccine. The polymeric mRNA vaccines were footpad subcutaneously injected into B16-OVA tumor-bearing mice to evaluate their anti-tumor efficacy.

### Example 1—The PHTA-Cn Series

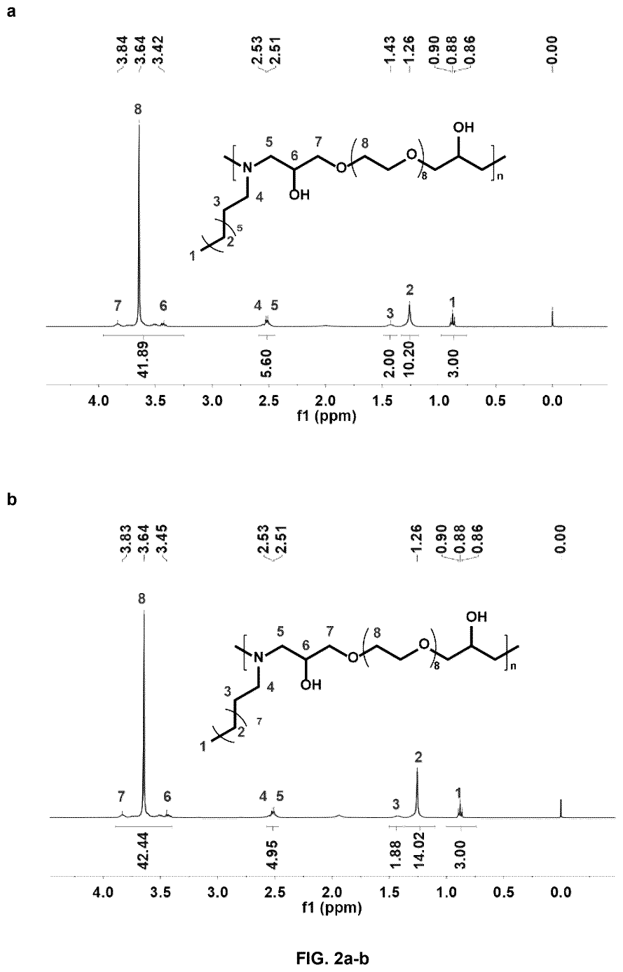

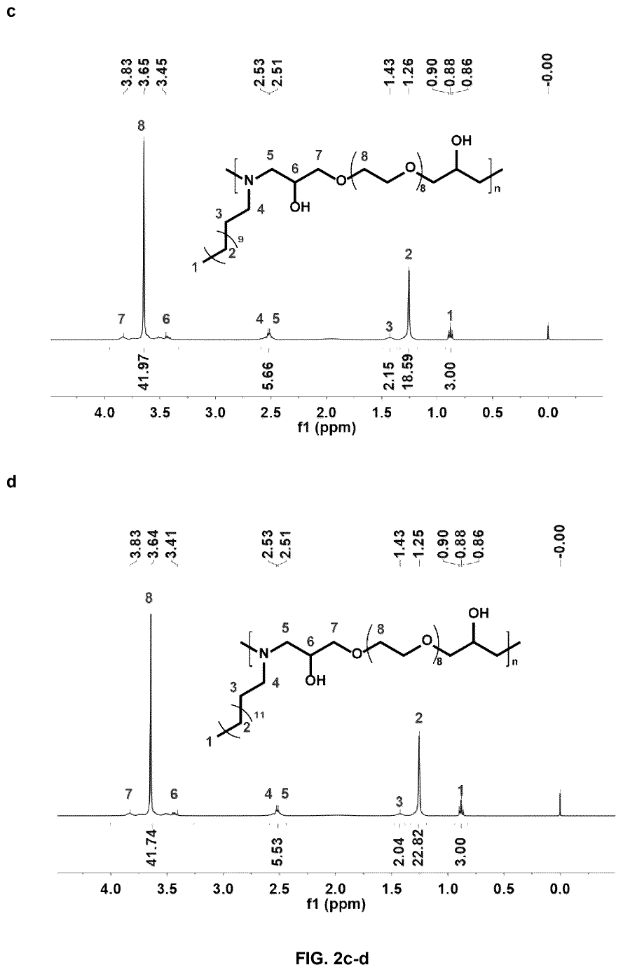

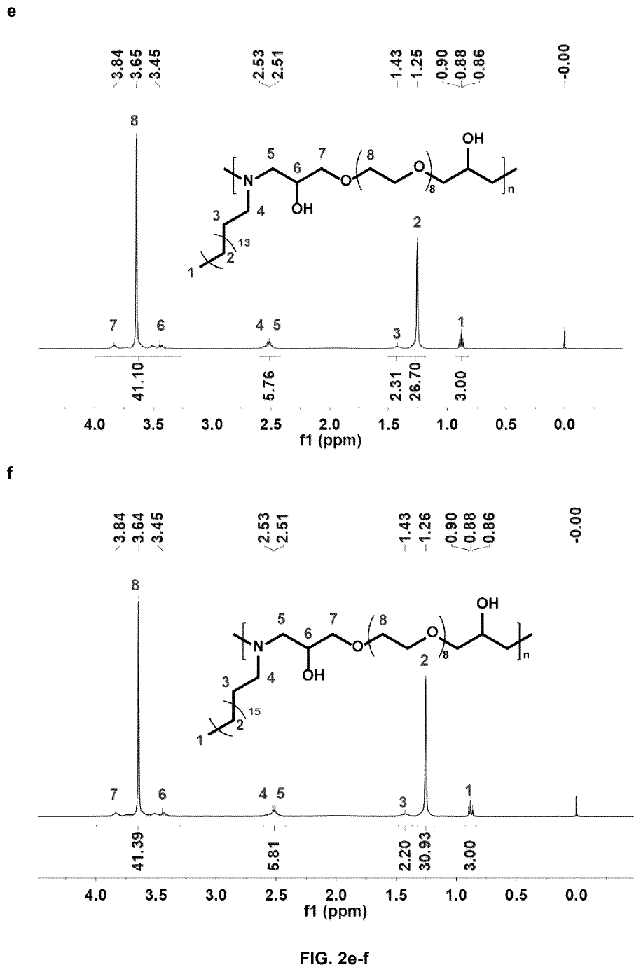

The synthesis of PHTA-Cn polymers was examined by ¹H NMR spectroscopy. The proton signals of repeating units and the integral ratios in line with the theoretical values in ¹H NMR spectra suggested the integrity of the chemical structures and successful synthesis of PHTA-Cn polymers (FIG. 2).

### Characterization of PHTA-Cn/mOVA Nanovaccines

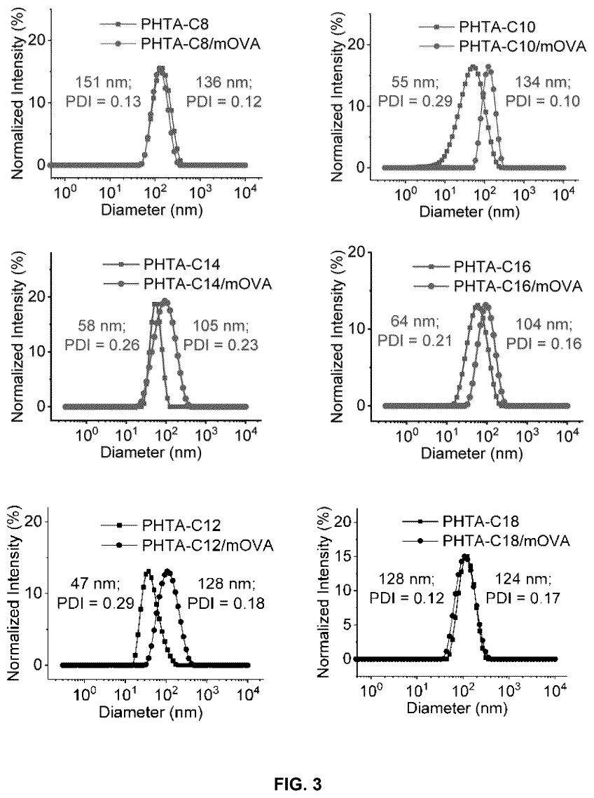

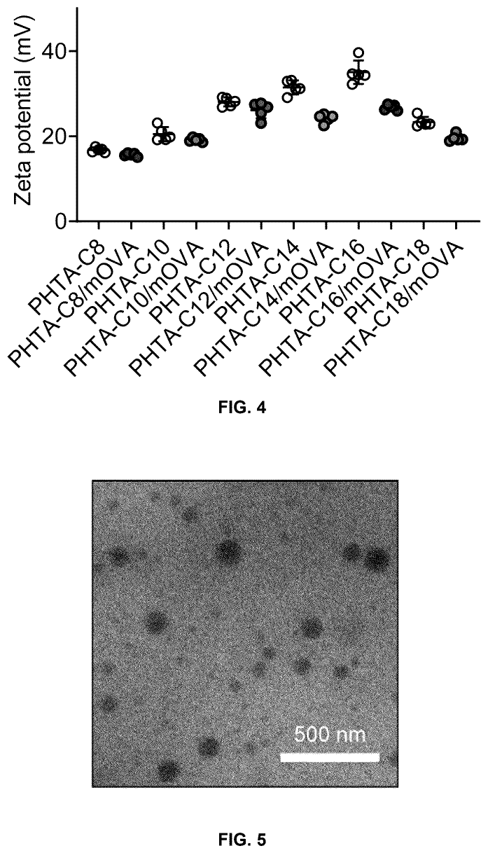

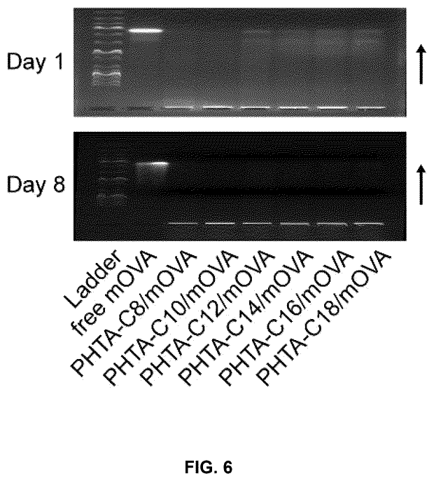

The PHTA-Cn/mOVA nanovaccines were constructed by formulating mRNA encoding OVA with PHTA-Cn polymers and cholesterol by microfluidic mixing. The physicochemical properties of PHTA-Cn/mOVA nanovaccines were characterized by DLS, zeta potential, and TEM. The DLS results showed that all the PHTA-Cn/mOVA nanovaccines were nanoparticles with diameters <200 nm and narrow distributions (<0.3) (FIG. 3). Compared with PHTA-Cn polymeric nanoparticles (PNPs), the decreased zeta potential of PHTA-Cn/mOVA complexes indicated successful loading of mOVA (FIG. 4). The representative TEM image showed nanospherical morphology of PHTA-based PNPs (FIG. 5). The mRNA binding capacities of the PHTA-Cn-based polymeric nanocarrieris were verified by agarose gel electrophoresis on day 1 and day 8 post mRNA loading. The immobile complexes remaining in the starting zone of the gel and no band for free mRNA indicated efficient loading of mRNA into polymeric nanocarriers (FIG. 6). These findings collectively suggested successful construction of PHTA-Cn/mOVA nanovaccines.

In Vitro mRNA Delivery Efficiency of PHTA-Cn/mGFP Complex

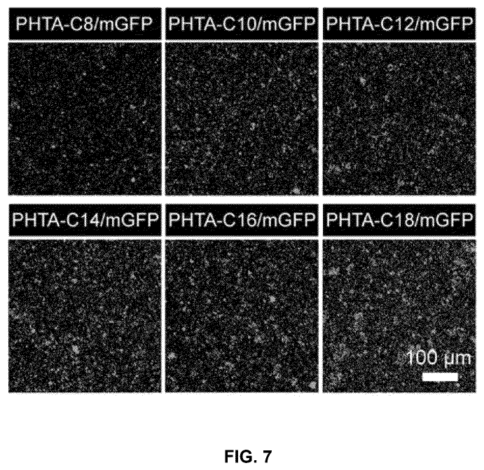

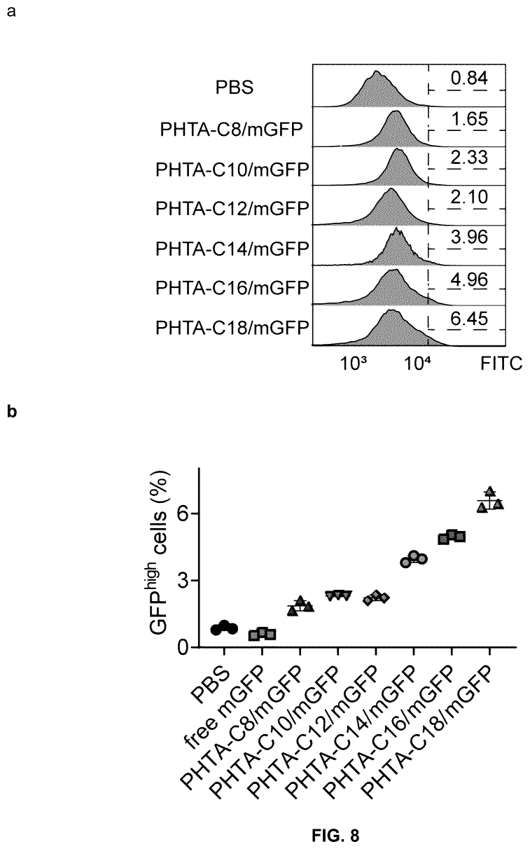

To evaluate in vitro mRNA delivery efficiency, different PHTA-Cn/mGFP complexes were prepared and their in vitro transfection efficiency in DC 2.4 cells were characterized by confocal microscopy images and flow cytometry. The fluorescence signals in confocal microscopy images showed all the polymeric nanocarriers exhibited different degrees of mRNA delivery ability and enabled successful GFP expression (FIG. 7). The GFP expression analyzed by flow cytometry showed that PHTA-C18/mGFP exhibited the highest GFP expression level (FIG. 8). It can be speculated that stronger hydrophobicity endowed by longer alkyl chain of PHTA-C18 polymer promoted the polymer self-assembly and improved the stability of PNPs through hydrophobic interactions, thus leading to higher mRNA transfection efficiency.

In Vivo mRNA Delivery Efficiency of PHTA-Cn/mFluc Complex

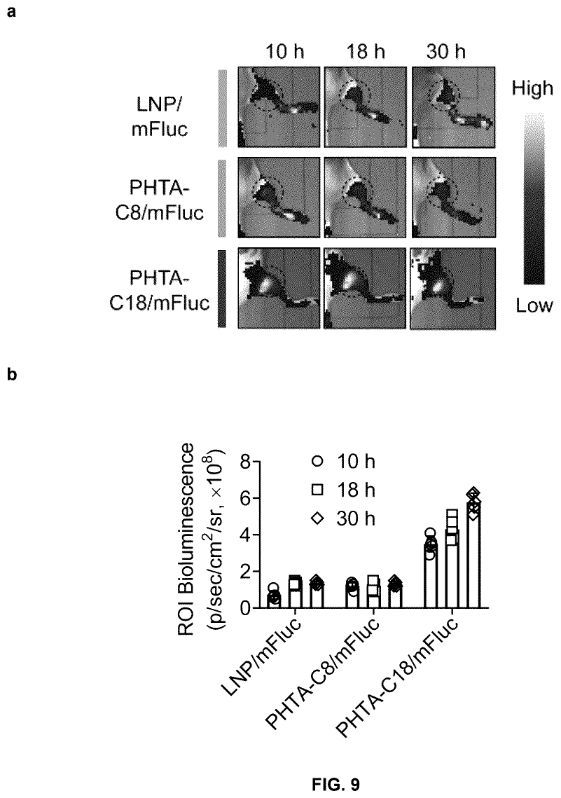

To evaluate in vivo mRNA delivery efficiency, PHTA-C18/mFluc and PHTA-C8/mFluc complex were prepared, footpad subcutaneously injected into C57BL/6 mice, and analyzed by IVIS optical imaging system. The bioluminescence images and corresponding quantitative results indicated PHTA-C18/mFluc treatment led to much stronger luminescence intensity due to the expression of luciferase protein than the PHTA-C8/mFluc-treated group (FIG. 9). These results demonstrated that PHTA-C18 PNP could promote effective mRNA migration to the lymph nodes (LNs) and allow sustained rapid protein translation in vivo.

### Proof of Concept Study of PHTA-Cn/mOVA Nanovaccine as a Therapeutic Cancer Vaccine

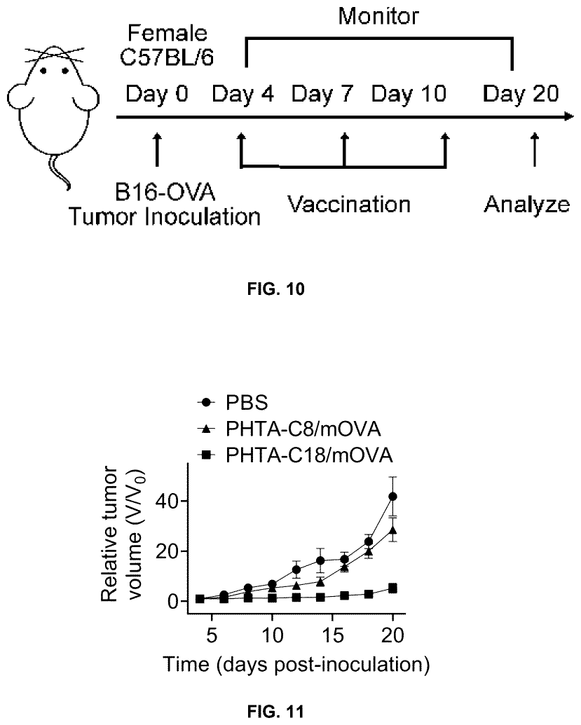

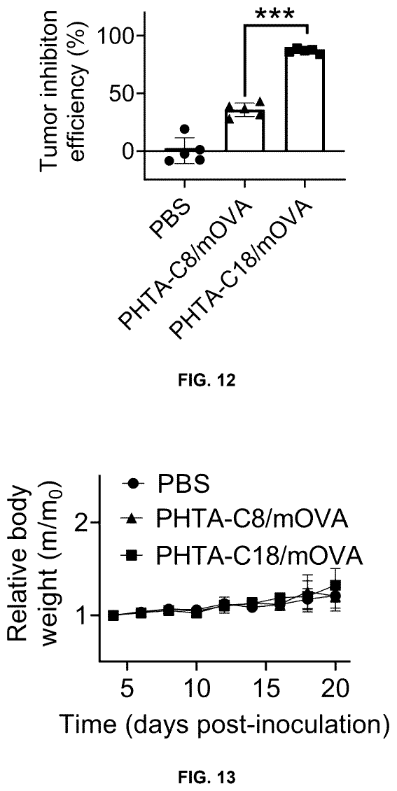

The antitumor effect of PHTA-C18/mOVA nanovaccine was assessed in the subcutaneous B16-OVA melanoma tumor model. On days 4, 7, and 10 post-inoculation, PHTA-C18/mOVA as well as PHTA-C8/mOVA and PBS as control formulations were subcutaneously injected into mice (FIG. 10). The antitumor efficacy was evaluated by measuring the tumor volume growth in different treatment groups. Mice vaccinated with PHTA-C8/mOVA showed a slight degree of tumor inhibition compared with PBS-treated group, whereas PHTA-C18/mOVA treatment generated sustained tumor suppression (FIG. 11). Correspondingly, the tumor inhibition efficiency of PHTA-C18/mOVA was calculated to be 87%, which is higher than that of PHTA-C8/mOVA (36%) (FIG. 12). The basically unchanged body weights of all the groups indicated that PHTA-based polymeric mRNA vaccines were well tolerated without apparent toxicity (FIG. 13).

### Example 2—the PHTA-BCn Series

### Characterization of PHTA-BCn Polymers

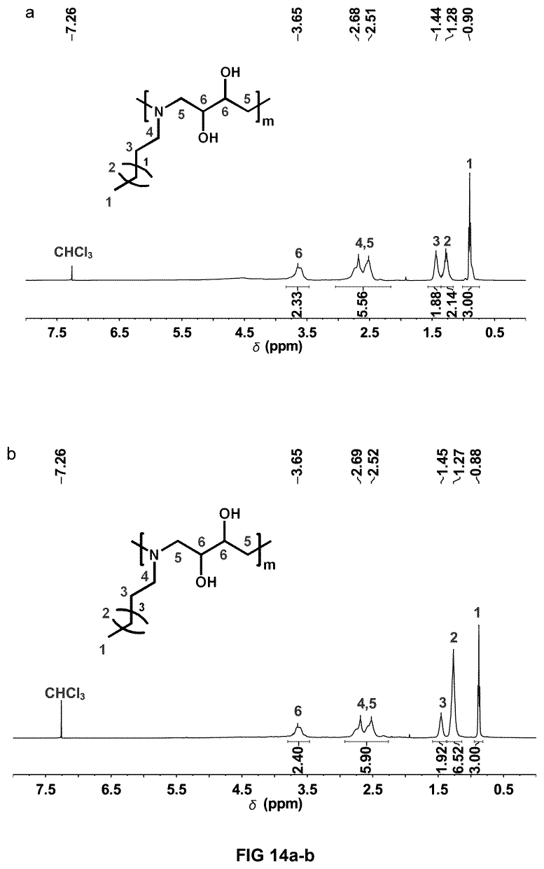

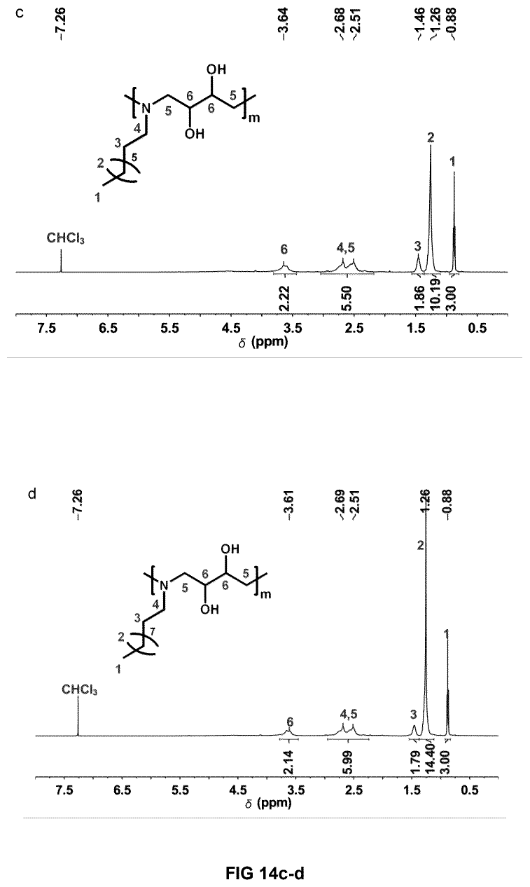

PHTA-BCn polymers were synthesized by amino-epoxy polymerization of 1,3-butadiene diepoxide (BDE) and different alkylamines, where “B” represents BDE monomer and “n” represents the number of carbon atoms on the polymer side chain. The synthesis of PHTA-BCn polymers was examined by ¹H NMR spectroscopy. The proton signals of repeating units and the integral ratios in line with the theoretical values in ¹H NMR spectra suggested successful synthesis of PHTA-BCn polymers (FIG. 14).

### Characterization of PHTA-BCn/mRNA Complex

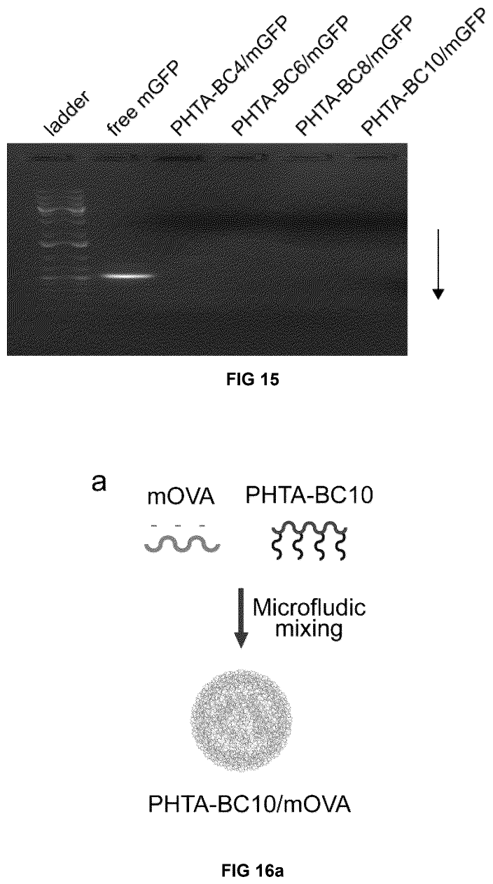

The PHTA-BCn/mGFP complexes were constructed by simple microfluidic mixing of PHTA-BCn polymers and mGFP. The mRNA loading capacities of PHTA-BCn polymeric nanocarriers were verified by agarose gel electrophoresis. As shown in the agarose gel electrophoresis image (FIG. 15), all the PHTA-BCn/mGFP complexes were retained in the starting zone of the gel, and no band for free mRNA was observed, indicating that PHTA-BCn-based polymeric nanocarrier could successfully load mGFP.

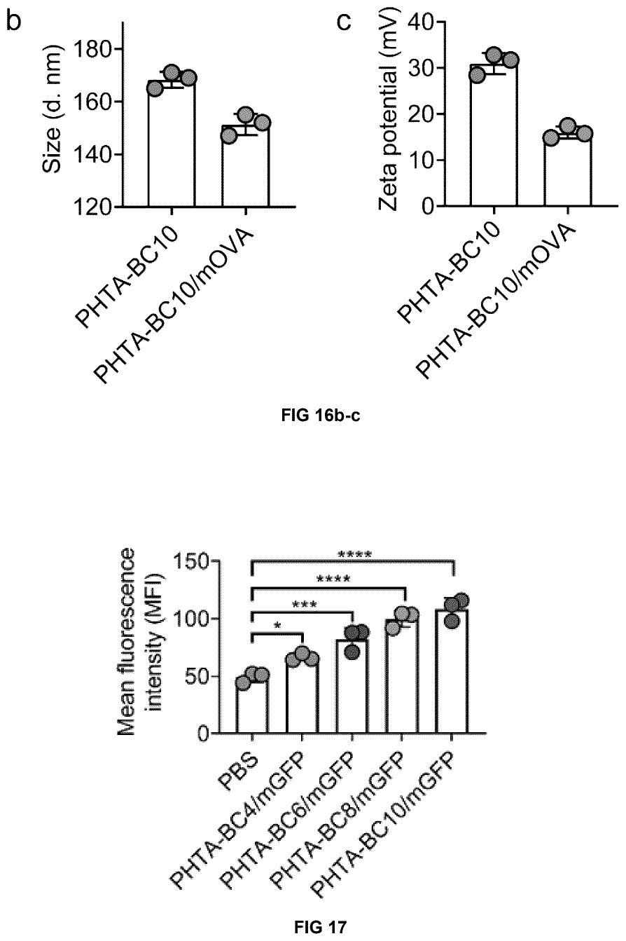

Representative PHTA-BC10/mOVA nanovaccine was constructed by microfluidic mixing of PHTA-BC10 polymer and mOVA (FIG. 16a). The physicochemical properties of PHTA-BC10/mOVA nanovaccine were characterized by DLS and zeta potential. The DLS results showed that PHTA-BC10/mOVA nanovaccines were nanoparticles with diameters <200 nm (FIG. 16b). The zeta potential of PHTA-BC10/mOVA nanovaccine was lower than that of PHTA-BC10 PNP without loading mRNA, further indicating successful loading of mOVA (FIG. 16c). These results confirmed successful loading of mOVA and construction of PHTA-BC10/mOVA nanovaccine.

In Vitro mRNA Delivery Efficiency of PHTA-BCn/mGFP Complex

To evaluate in vitro mRNA delivery efficiency of PHTA-BCn-based polymeric nanocarriers, PHTA-BCn/mGFP complexes were prepared by loading mGFP and investigated for their transfection efficiency in DC 2.4. As indicated by semi-quantitative results of mean fluorescence intensity (MFI) of green fluorescent protein (GFP), the DC 2.4 treated with different PHTA-BCn/mGFP complexes showed different degrees of GFP expression, confirming successful in vitro mRNA delivery ability of PHTA-BCn-based polymeric nanocarriers (FIG. 17).

### Proof of Concept Study of PHTA-BCn/mOVA Nanovaccine as a Therapeutic Cancer Vaccine

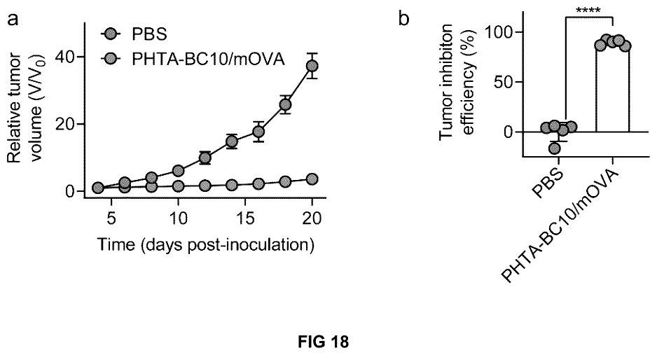

The antitumor effect of representative PHTA-BC10/mOVA nanovaccine was evaluated in the subcutaneous B16-OVA melanoma tumor model. On days 4, 7, and 10 post-inoculation, PHTA-BC10/mOVA nanovaccine and PBS as control formulation were footpad subcutaneously injected into mice. The antitumor efficacy was evaluated by measuring the tumor volume growth in different treatment groups. The results indicated that mice vaccinated with PHTA-BC10/mOVA nanovaccine generated effective and sustained tumor suppression compared with PBS-treated mice (FIG. 18a). The tumor inhibition efficiency of PHTA-BC10/mOVA nanovaccine was calculated to be 89% (FIG. 18b). These results demonstrated the potential of PHTA-BCn-based polymeric nanocarrier for mRNA therapeutics delivery.

### Example 3—the PHTA-BFn Series

### Characterization of PHTA-BFn Polymers

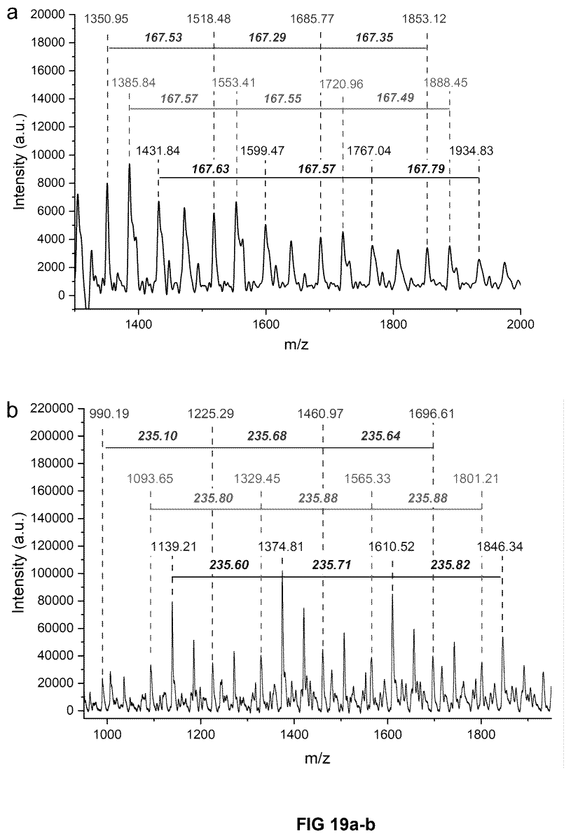

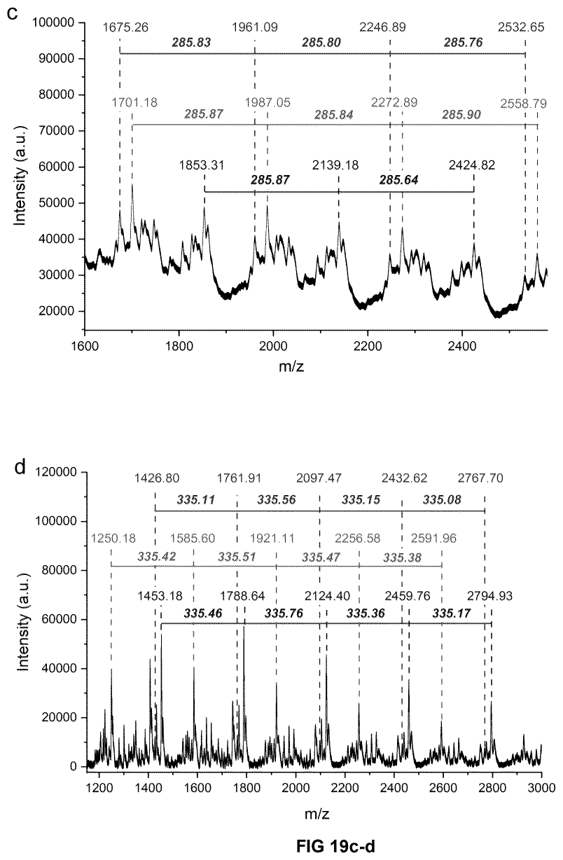

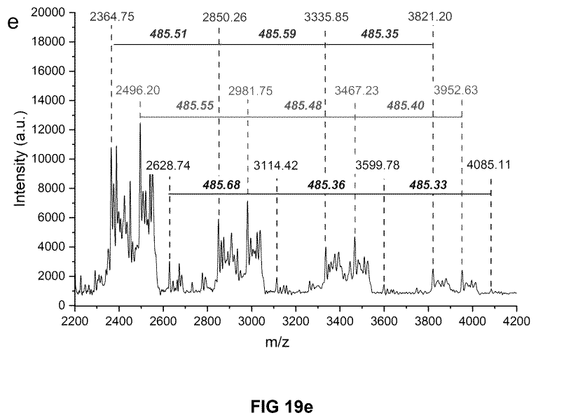

The PHTA-BFn polymers were synthesized by amino-epoxy polymerization of 1,3-butadiene diepoxide (BDE) and different fluorine-substituted alkylamines, where “B” represents BDE monomer and “n” represents the number of fluorine atoms on the polymer side chain. The synthesis of PHTA-BFn polymers was characterized by matrix-assisted laser desorption/ionization time of flight mass spectrometry (MALDI-TOF-MS). As shown in the MALDI-TOF-MS spectra (FIG. 19), there are three series of peaks with same m/z intervals consistent with the molar mass of PHTA-BFn polymer repeating unit in each MALDI-TOF-MS spectrum, indicating successful synthesis of alternating copolymers PHTA-BFn.

### Characterization of PHTA-BF7/mRNA Complex

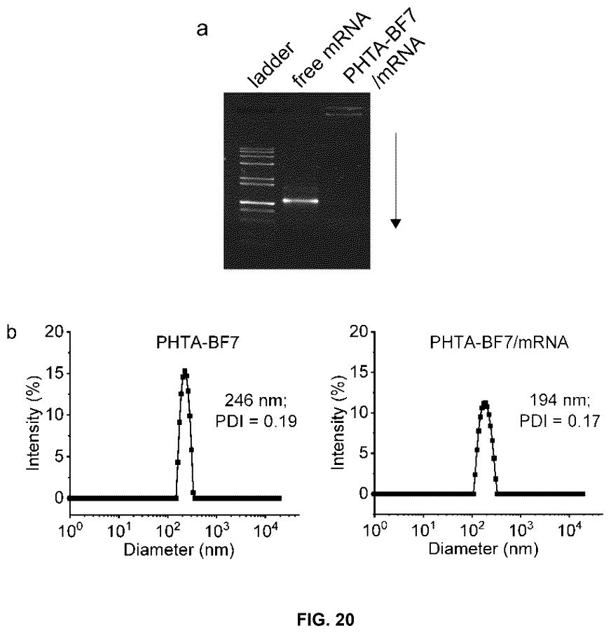

Representative PHTA-BF7/mRNA complex was constructed by microfluidic mixing of PHTA-BF7 polymer and mRNA. The mRNA loading capacity of PHTA-BF7 polymeric nanocarrier was verified by agarose gel electrophoresis. As shown in the agarose gel electrophoresis image (FIG. 20a), the PHTA-BF7/mRNA complex was retained in the starting zone of the gel, and no band for free mRNA was observed, indicating that PHTA-BF7-based polymeric nanocarrier could successfully load mRNA. The DLS results showed that PHTA-BF7/mRNA complex was nanoparticle with diameter <200 nm and narrow distribution (FIG. 20b). These results suggested successful construction of PHTA-BF7/mRNA complex.

In Vitro mRNA Delivery Efficiency of PHTA-BF7/mGFP Complex

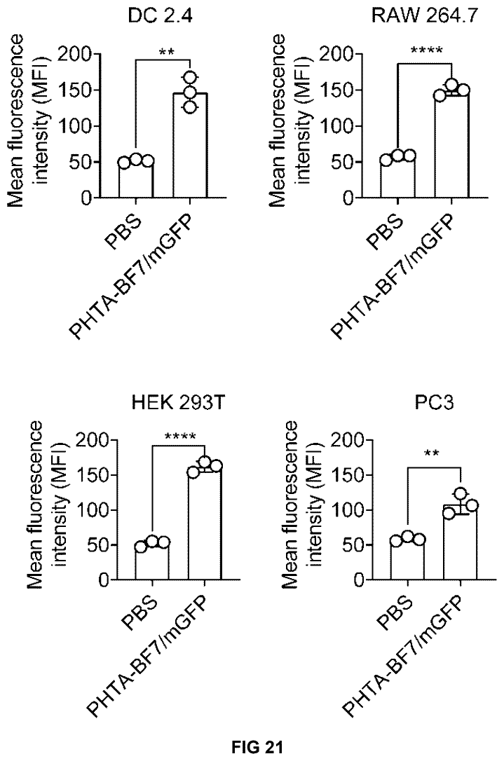

In vitro mRNA delivery efficiency of PHTA-BF7/mGFP complex was investigated in antigen-presenting cell DC 2.4, macrophage RAW 264.7, and tumor cells HEK 293T and PC3. As indicated by semi-quantitative results of mean fluorescence intensity (MFI) of green fluorescent protein (GFP) (FIG. 21), all these four cells exhibited efficient GFP protein expression after treatment of PHTA-BF7/mGFP complex. These results suggested that PHTA-BF7-based polymeric nanocarriers could successfully deliver mRNA in vitro.

In Vivo mRNA Delivery Efficiency of PHTA-BF7/mFluc Complex

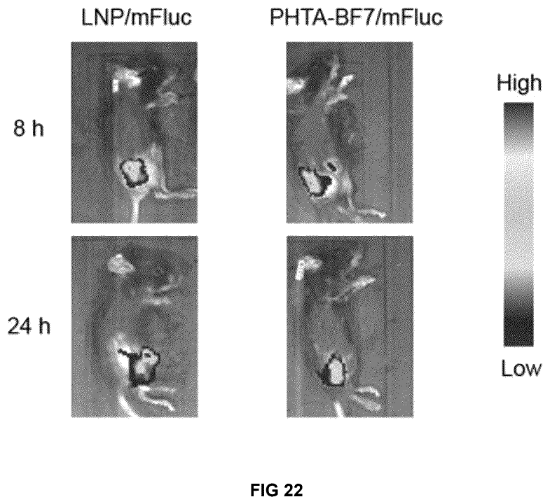

To further investigate in vivo mRNA delivery efficiency of PHTA-BF7/mFluc complex, PHTA-BF7/mFluc complex was subcutaneously injected into mice and characterized by IVIS optical imaging system at 8 h and 24 h post injection. As indicated by bioluminescence images (FIG. 22), PHTA-BF7/mFluc treatment induced effective luciferase protein expression in mice, demonstrating the in vivo mRNA delivery ability of PHTA-BFn-based polymeric nanocarrier.

### CONCLUSIONS

In conclusion, a new kind of ionizable alternating copolymers PHTA with hydroxyl tertiary amine (HTA) repeating units has been developed for polynucleotide delivery. PHTA polymers were synthesized by amino-epoxy polymerization of diepoxide monomer 1 and amine monomer 2. By changing the types of diepoxide monomer 1 and amine monomer 2, three representative series of PHTA polymers (i.e., PHTA-Cn, PHTA-BCn, and PHTA-BFn) were synthesized and studied for polynucleotide delivery efficiency. The result showed that all these three series of PHTA polymers could successfully deliver polynucleotides into cells in vitro and in vivo.

1. An ionizable polymer comprising a constitutional unit according to formula (I): 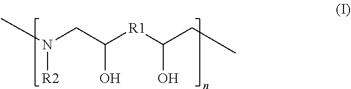
wherein
R1 represents a covalent bond or a linking moiety derived from a polyalkylene glycol; and
R2 represents a linear or branched aliphatic hydrocarbyl or a linear or branched fluorinated aliphatic hydrocarbyl group.

2. The ionizable polymer of claim 1, wherein the polyalkylene glycol is a polyethylene glycol.

3-5. (canceled)

6. The ionizable polymer of claim 1, wherein R2 is selected from the group consisting of butyl, hexyl, octyl, decyl, dodecyl, tetradecyl, hexadecyl and octadecyl.

7. The ionizable polymer of any one of claim 1, wherein R2 is selected from the group consisting of difluoroethyl, pentafluoropropyl, heptafluorobutyl, nonafluoropentyl, and pentadecafluorooctyl.

8. (canceled)

9. The ionizable polymer of claim 1, wherein R1 is according to formula (II) 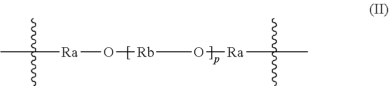
wherein,
each Ra is independently a covalent bond or a linear —C₁₋₆alkylene-;
Rb is —C₁₋₆alkylene-, said alkylene being a linear alkylene and optionally substituted by one or more groups selected from OH, NH₂, C₁₋₄alkyl, and halogen;
p is an integer from 1 to 10.

10. The ionizable polymer of claim 1 wherein each Ra is —C₁alkylene-, and Rb is a linear and unsubstituted —C₂alkylene-.

11. The ionizable polymer of claim 1, wherein the ionizable polymer comprises a constitutional unit selected from the group consisting of:

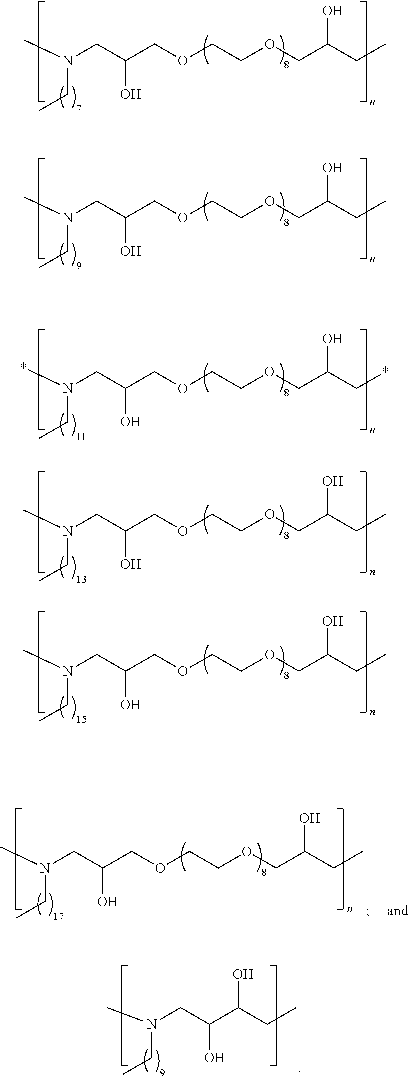

12. The ionizable polymer of claim 1, wherein the ionizable polymer comprises a constitutional unit selected from the group consisting of:

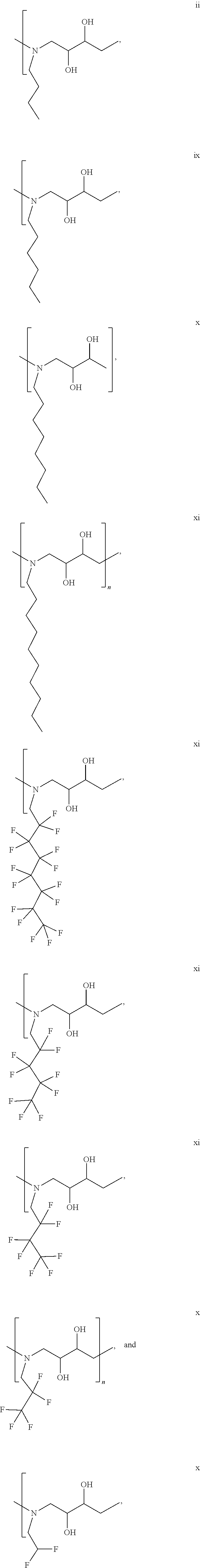

13. A composition comprising the ionizable polymer of claim 1, and a polynucleotide, wherein the polynucleotide is complexed with polymeric nanoparticles (PNP) formed from said ionizable polymer.

14. The composition of claim 13, wherein the polynucleotide is selected from the group comprising RNA and DNA, optionally wherein the polynucleotide is mRNA or siRNA.

15. The composition of claim 13, wherein:
a) R1 is derived from a polyalkylene glycol and the polynucleotide-PNP complexes are formed by mixing polynucleotide, cholesterol and ionizable polymer, optionally wherein the polyalkylene glycol is polyethylene glycol; or
b) R1 is a covalent bond and the polynucleotide-PNP complexes are formed by mixing polynucleotide and ionizable polymer.

16. The composition of claim 13, wherein the polynucleotide-PNP complex is capable of delivering said polynucleotide into a human or non-human animal cell, optionally wherein the polynucleotide is mRNA and/or wherein the human cell is a cancer cell.

17. The composition of claim 13, wherein the polynucleotide is mRNA or DNA, and where the mRNA or DNA encodes a cancer-specific antigen, an infectious disease-specific antigen, or a therapeutic protein.

18. A pharmaceutical composition comprising the composition of claim 13.

19. The pharmaceutical composition of claim 18, comprising polymeric nanoparticles (PNP) formed from a polynucleotide and an ionizable polymer, wherein:
a) R1 is a linking moiety derived from a polyethylene glycol and R2 is octadecyl, or
b) R1 is a covalent bond and R2 is decyl.

20.-23. (canceled)

24. A method of prophylaxis or treatment of a subject, comprising administering to the subject an efficacious amount of:
a composition of claim 13 in which the polynucleotide is an mRNA or DNA.

25. The method of claim 24, wherein one of the following applies:
(a) the treatment is of a cancer in a subject and the mRNA or DNA encodes a cancer-specific antigen or a therapeutic protein;
(b) the prophylaxis is of a virus-related infection in a subject and the mRNA or DNA encodes a virus-specific antigen; or
(c) the treatment or prophylaxis is of a protein-deficiency disorder in a subject, wherein the mRNA or DNA encodes a protein or peptide that is absent or non-functional in the subject.

26.-29. (canceled)

30. A method of producing a composition according to claim 13 in which the polynucleotide is mRNA or DNA, the method comprising the steps:
i) combining a monomer of formula (III) and a monomer of formula (IV) through amino-epoxy ring-opening polymerization to form an ionizable polymer comprising a constitutional unit according to formula (I); and
ii) mixing the ionizable polymer with an mRNA or DNA which encodes an antigenic polypeptide or a therapeutic protein,
wherein, 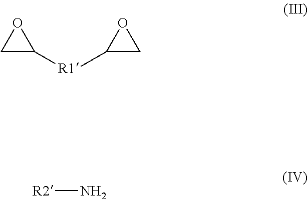
wherein,
R1′ represents a covalent bond or a polyalkylene glycol moiety; and
R2′ represents a linear or branched aliphatic hydrocarbyl or a linear or branched fluorinated aliphatic hydrocarbyl group.

31. A method of prophylaxis or treatment of a subject, comprising administering to the subject an efficacious amount of:
a pharmaceutical composition of claim 18.

32. The method of claim 31, wherein one of the following applies:
(a) the treatment is of a cancer in a subject and the mRNA or DNA encodes a cancer-specific antigen or a therapeutic protein;
(b) the prophylaxis is of a virus-related infection in a subject and the mRNA or DNA encodes a virus-specific antigen; or
(c) the treatment or prophylaxis is of a protein-deficiency disorder in a subject, wherein the mRNA or DNA encodes a protein or peptide that is absent or non-functional in the subject.
